(19) United States
(12) Patent Application Publication
(10) Pub. No.: US 2025/0214928 A1
(43) Pub. Date: Jul. 3, 2025

(54) Process for the preparation of marimastat, marimastat prepared by this process, a pharmaceutical composition comprising the same, and uses thereof

(71) Applicant: PIKRALIDA SPÓLKA Z OGRANICZONA ODPOWIEDZIALNOSCIA, Poznan (PL)

(72) Inventors: Stanislaw PIKUL, Ceradz Dolny (PL); Marek MASNYK, Warszawa (PL); Katarzyna SIDORYK, Warszawa (PL); Lukasz MUCHA, Warszawa (PL); Marta MAGDYCZ, Dabrowa Chotomowska (PL); Sylwia PIASECKA, Duchnice (PL)

(21) Appl. No.: 19/001,776

(22) Filed: Dec. 26, 2024

(30) Foreign Application Priority Data
Dec. 29, 2023 (PL) P.447395

(51) Int. Cl. C07C 235/04; A61K 9/20; A61K 31/16; C07C 231/02; C07C 231/10; C07C 231/24

(52) U.S. Cl. CPC C07C 235/04; A61K 9/2095; A61K 31/16; C07C 231/02; C07C 231/10; C07C 231/24

(57) ABSTRACT

The invention relates to a process for the preparation of marimastat. The invention further relates to marimastat prepared by the process, and a pharmaceutical composition comprising said marimastat. The invention also relates to the pharmaceutical composition for use as a medicament, and said marimastat for use as a medicament. The invention further relates to the pharmaceutical composition for use in the prevention and treatment of diseases associated with hyperactivity of extracellular matrix metalloproteinases, and said marimastat for use in the prevention and treatment of diseases associated with hyperactivity of extracellular matrix metalloproteinases.

### FIELD OF THE INVENTION

The present invention relates to a process for the preparation of marimastat, marimastat prepared by this process, a pharmaceutical composition comprising the same, and their uses in the prevention and treatment of diseases associated with hyperactivity of extracellular matrix metalloproteinases.

### BACKGROUND OF THE INVENTION

Marimastat, or (2R,3S)-N-1-((S)-3,3-dimethyl-1-methylamino-1-oxobutan-2-yl)-N-4,3-dihydroxy-2-isobutylsuccinamide, is a compound with the structural formula:

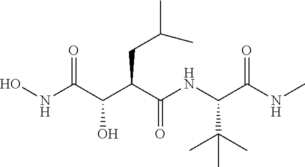 [SMILES: CNC(=O)[C@@H](NC(=O)[C@H](CC(C)C)[C@H](O)C(=O)NO)C(C)(C)C]

Marimastat is an inhibitor of extracellular matrix metalloproteinases (MMPs). Extracellular matrix metalloproteinases belong to a family of enzymes involved in the degradation of the extracellular matrix, which forms the tissue skeleton and allows the cell to exchange information with the environment. Excessive activity of extracellular matrix metalloproteinases causes destruction of the extracellular matrix, which results in improper functioning of the cell, which in turn contributes to the development of many diseases. Excessive activity of extracellular matrix metalloproteinases occurs, among others, in arthritis and periodontitis, atherosclerosis and cardiovascular diseases [1, 2, 3]. Metalloproteinases play an important role in the development and severity of vascular complications in diabetic patients [4]. They are believed to be involved in cell differentiation, migration and apoptosis, as well as in angiogenesis [3]. Metalloproteinases play an important role in carcinogenesis and metastasis. Their activity is correlated with the degree of malignancy of tumors and the tendency to metastasize [1, 2, 3]. Increased activity of extracellular matrix metalloproteinases is found in Alzheimer's disease, multiple sclerosis, amyotrophic lateral sclerosis, multiple myeloma and malignant melanoma [10, 11]. It has been proven that marimastat crosses the blood-brain barrier and has an inhibitory effect on the activity of metalloproteinases in the brain. In a mouse study, marimastat reduced the duration and number of seizures in a kainic acid-induced status epilepticus model [5]. Marimastat is also important in the treatment of advanced pancreatic cancer [6, 7, 8, 9]. Marimastat has been tested in over 400 patients in phase I/II trials in a wide variety of solid tumors. Most of these studies measured tumor-specific antigens (CEA in CRC, CA 19/9 in pancreatic cancer, CA 125 in ovarian cancer, prostate-specific antigen in prostate cancer) as surrogate markers of biological activity. A meta-analysis of all these studies, as well as analyzes of individual studies, showed that marimastat treatment significantly reduced all four growth indicators of tumor-specific antigens in a dose-dependent manner [9].

In the Polish patent description PL 174279 B1, example II discloses that marimastat (PKL-021) was prepared in a manner analogous to the process described in example I and NMR data for marimastat were provided. In the following Scheme 1, the reaction conditions, yields and methods of purification of intermediate products (chromatographic columns) are given for the compound described in example I, in analogy to which marimastat was obtained.

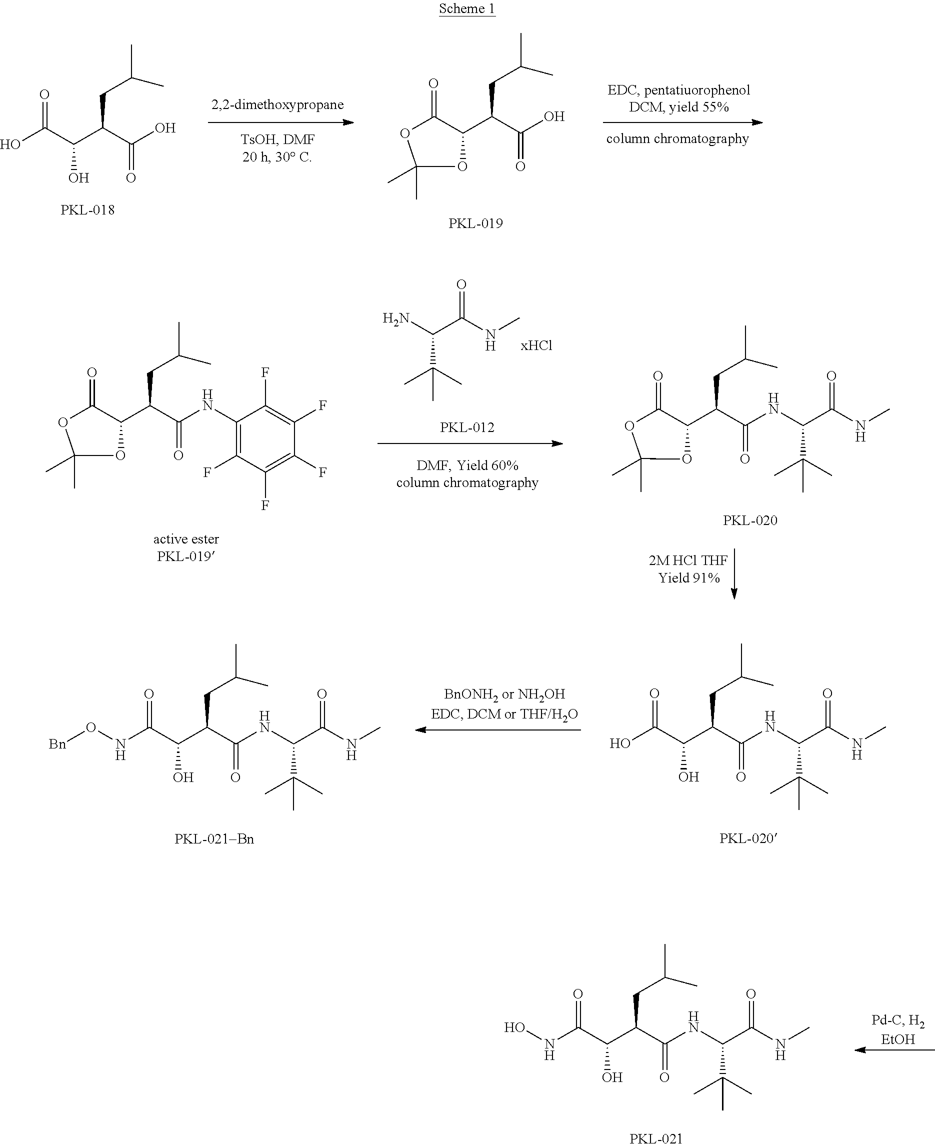

Thus, PL 174279 B1 does not disclose the exact procedures, the yield of the entire process as well as the purity of the obtained marimastat.

The publication Richard J Davenport, Robert J Watson: “An improved synthesis of the broad-spectrum matrix metalloprotease inhibitor marimastat”; Tetrahedron Letters, Volume 41, Issue 41, Jul. 10,2000, pp. 7983-7986 discloses a process for preparing marimastat. The synthesis process described in the publication includes a detailed description only of the last step of the synthesis, i.e., the reaction leading to the preparation of marimastat. In the first step of the synthesis, only the reagents used to carry out the reaction were given (2,2-dimethoxypropane, in the presence of p-toluenesulfonic acid), but the temperature at which the reaction was carried out, the reaction duration and the method of product isolation were not given. The second step of the synthesis involved the direct coupling of carboxylic acid (PKL-019) with L-tert-leucine methylamide (PKL-012) using EDC. PKL-020 was obtained with a yield of 60% after maceration in diethyl ether. There is no data on the reaction duration, temperature, base used, processing of the reaction mixture and purity of the obtained PKL-020. In the last step of the synthesis, PKL-020 was reacted with unblocked hydroxylamine (NH₂OH) in tetrahydrofuran (THF) at boiling. The publication particularly highlights the advantageous use of this solvent instead of dimethylformamide (DMF). Thereby, marimastat was obtained with a yield of 93% and a purity of 99.6-99.8%. The total yield of the marimastat synthesis process was 56%. However, the publication lacks detailed procedures for the earlier steps. Only general reaction conditions and yields are given, but no information about the purity of intermediate compounds, especially PKL-020. Scheme 2 of the synthesis of marimastat according to the Tetrahedron Letters publication is shown below.

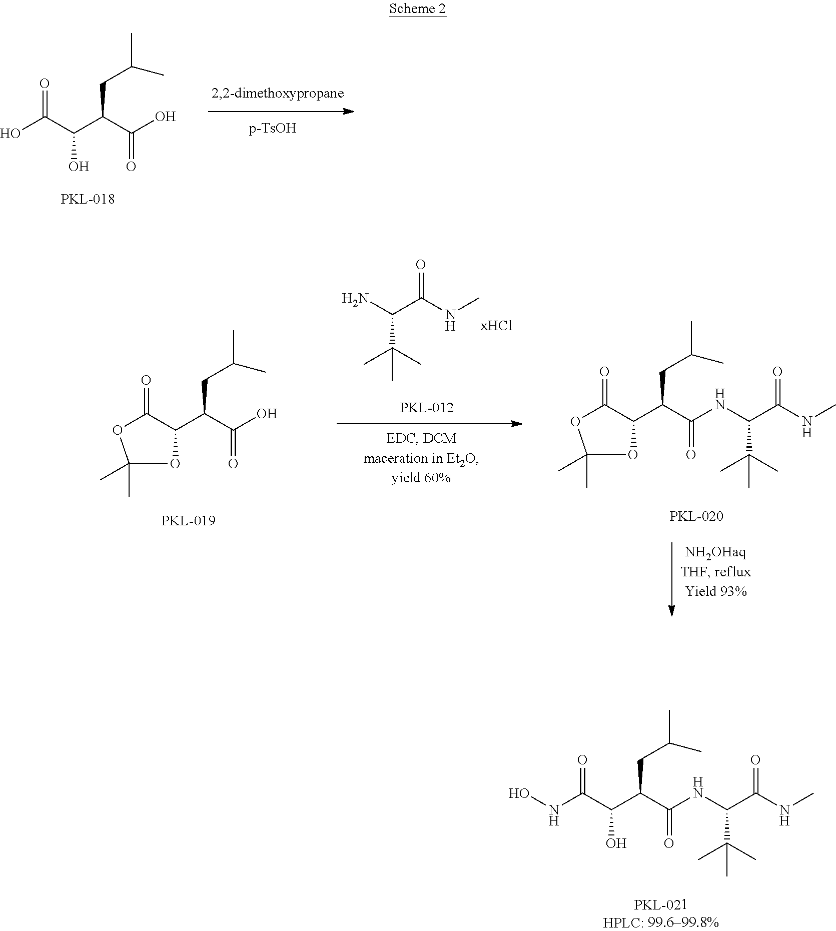

The publication Kai Jenssen, Katherina Sewald and Norbert Sewald: “Synthesis of Marimastat and a Marimastat Conjugate for Affinity Chromatography and Surface Plasmon Resonance Studies”; Bioconjugate Chem. 2004, 15, 594-600 discloses the preparation of marimastat in a 5-step synthesis using PKL-018 and PKL-012 as substrates. A change in relation to the Polish patent description PL 174279 B1 and the Tetrahedron Letters (2000) publication is the use of hexafluoroacetone to protect alpha-hydroxy acid (PKL-018), which forms a dioxolane ring (PKL-019″) when reacted in DMSO. Then, the free acid group is activated by forming acid chloride (PKL-019″-CI), which is reacted with PKL-012 at a temperature of −65° C. The publication pointed out that a higher temperature leads to opening of the dioxolane ring and obtaining PKL-020′ with very low yield. Moreover, the intermediate PKL-020′ is a relatively unstable compound (unstable dioxolane ring), so purification is impossible. The derivative PKL-020′ is then converted with 85% yield (after chromatographic purification) into the stable intermediate PKL-021-Bn in reaction with the benzyl derivative of hydroxylamine (BnO-NH₂). The last step, leading to the preparation of marimastat, involves deprotection of the benzyl group, as described in the basic patent. The obtained crude marimastat (PKL-021) is then purified by preparative HPLC. Scheme 3 of the synthesis of marimastat according to the Bioconjugate Chem publication is shown below.

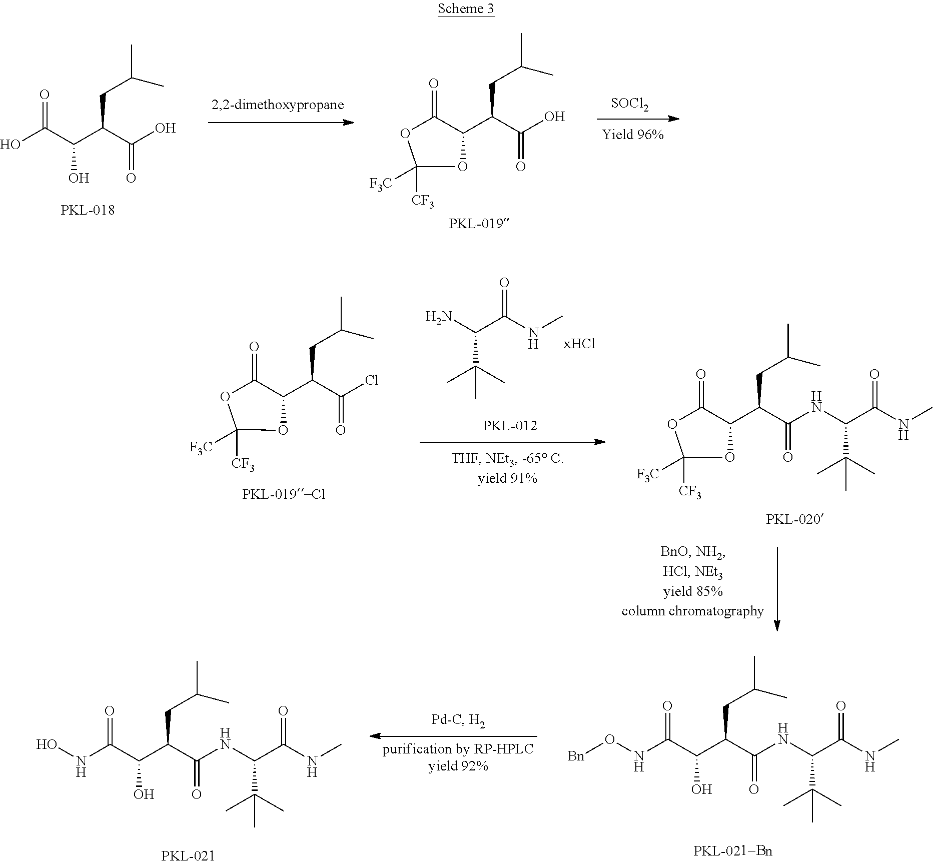

The publication Daniel E. Levy, France Lapierre, Weisheng Liang, Wenqing Ye, Christopher W. Lange, Xiaoyuan Li, Damian Grobelny, Marie Casabonne, David Tyrrell, Kevin Holme, Alex Nadzan and Richard E. Galardy: “Matrix Metalloproteinase Inhibitors: A Structure-Activity Study”; J. Med. Chem. 1998, 41, 199-223 discloses a 5-step synthesis of marimastat. Similarly to previous reports (PL 174279 B1, Tetrahedron Letters 2000), the acid group of PKL-018 is protected in the form of isopropylidene protection (PKL-019), then the intermediate product PKL-020 is obtained in a coupling reaction with PKL-012. After deprotection of the protecting group, the obtained PKL-020′ is reacted with diazomethane in methanol, which is converted to the final PKL-021 by reaction with hydroxylamine with a yield of 55%. The publication lacks the characteristics of intermediate compounds, except for the final compound PKL-021. Moreover, the explosive and highly toxic diazomethane is used in the synthesis. The general procedures given also state that the intermediate compounds are purified by column chromatography. Scheme 4 of the synthesis of marimastat according to the J. Med. Chem. publication is shown below.

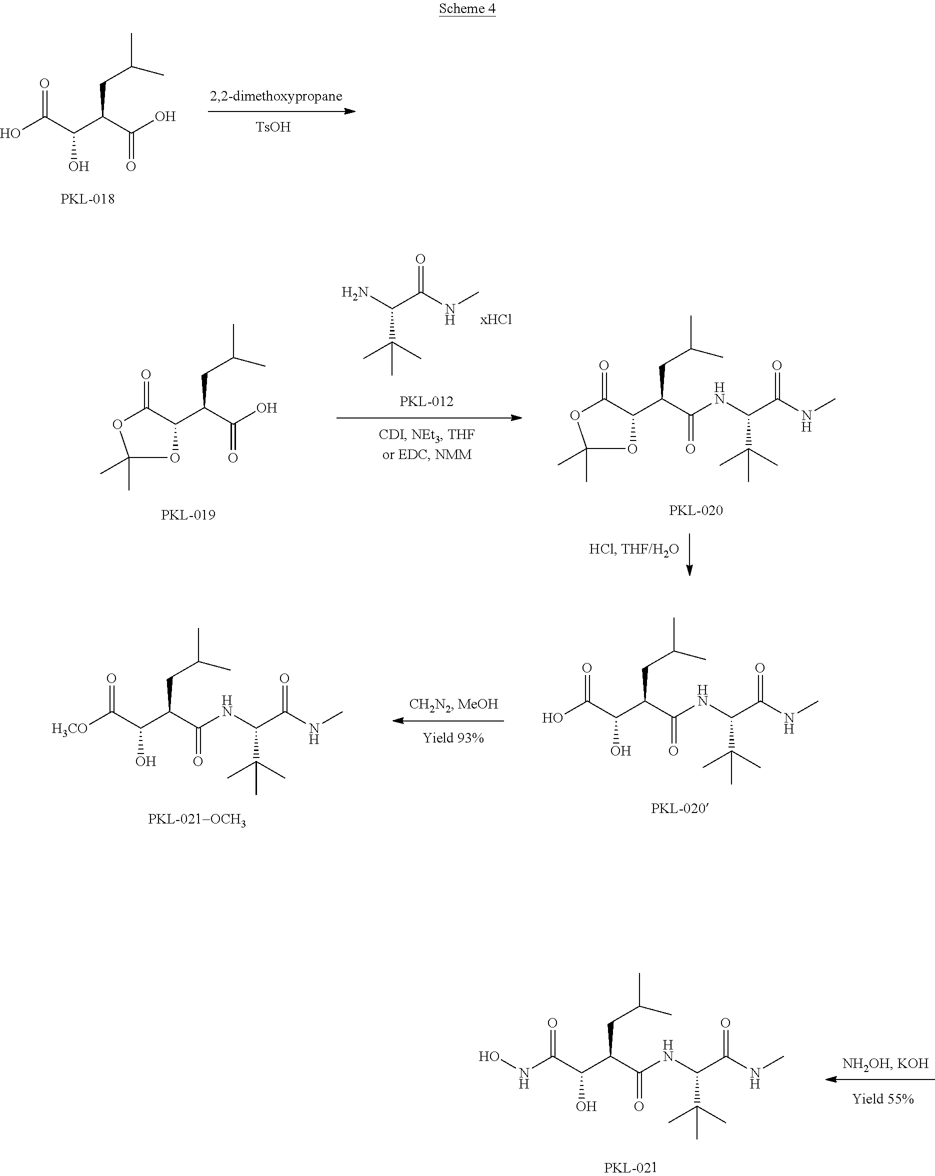

### SUMMARY OF THE INVENTION

The purpose of the invention is to develop a process for the preparation of marimastat (PKL-021) with high chemical and enantiomeric purity that meets the requirements for use in pharmaceutical formulation.

The subject of the invention is a process for the preparation of marimastat, characterized in that the process includes the following steps:
a) (2S,3R)-2-hydroxy-3-isobutylsuccinic acid (PKL-018) is reacted in the environment of 2,2-dimethoxypropane with the addition of p-toluenesulfonic acid (p-TsOH), pyridinium p-toluenesulfonate (PPTS) or 10-camphorsulfonic acid (CSA) at a temperature of 35-55° C. for 15-30 h, then after the reaction is completed, a tertiary amine selected from the group comprising diisopropylethylamine, triethylamine and N-methylmorpholine is added to obtain (R)-2-((S)-2,2-dimethyl-5-oxo-1,3-dioxolan-4-yl)-4-methylpentanoic acid (PKL-019); b) (R)-2-((S)-2,2-dimethyl-5-oxo-1,3-dioxolan-4-yl)-4-methylpentanoic acid (PKL-019) is reacted with (S)-2-amino-N,3,3-trimethylbutanamide or its hydrochloride (PKL-012) in the environment of 2-(1H-benzotriazole-1-yl)-1,1,3,3-tetramethylaminium tetrafluoroborate (TBTU) and a tertiary amine selected from the group comprising diisopropylethylamine, triethylamine and N-methylmorpholine in acetonitrile or methylene chloride, then the reaction mixture is subjected to an extraction process to obtain crude (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020), which is then used in step c) or macerated at boiling in methyl tert-butyl ether or isopropyl ether for 1-3 h, and then at room temperature for 15-30 h to obtain (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020); and c) (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020) or crude (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020) is reacted with a 50% aqueous solution of hydroxylamine in a solvent selected from a lower carboxylic acid ester, a non-polar solvent or a polar solvent, then acetone is added and stirred at boiling for 0.5-1 h, then the reaction mixture is subjected to azeotropic distillation process in order to remove water, and then stirred at room temperature for 15-30 h to obtain marimastat.

Preferably, the temperature in step a) is 40° C.

Preferably, the reaction in step b) is carried out in acetonitrile. Then, it is preferable that the extraction process in step b) includes diluting the reaction mixture with ethyl acetate, washing with 5% KHSO₄, saturated solution of NaHCO₃ and brine, drying over anhydrous Na₂SO₄ and evaporating the solvent.

Preferably, the reaction in step b) is carried out in methylene chloride. Then, it is preferable that the extraction process in step b) includes washing the reaction mixture with 5% KHSO₄, saturated solution of NaHCO₃ and brine, drying over anhydrous Na₂SO₄, and evaporating the solvent.

Preferably, the reaction in step b) is carried out at a temperature from −10° C. to room temperature.

Preferably, the reaction in step b) is carried out for 1-24 h.

Preferably, the tertiary amine in step b) is added in two portions, the first portion before adding (S)-2-amino-N,3,3-trimethylbutanamide or its hydrochloride (PKL-012), the second portion after adding (S)-2-amino-N,3,3-trimethylbutanamide or its hydrochloride (PKL-012).

Preferably, the tertiary amine is diisopropylethylamine.

Preferably, the reaction in step c) with the 50% aqueous solution of hydroxylamine is carried out at a temperature from room temperature to boiling point.

Preferably, the reaction in step c) with the 50% aqueous solution of hydroxylamine is carried out for 1-30 h.

Preferably, the lower carboxylic acid ester is selected from the group comprising ethyl acetate and isopropyl acetate.

Preferably, the non-polar solvent is selected from the group comprising 2-methyltetrahydrofuran, 1,2-dimethoxyethane, methylene chloride and chloroform.

Preferably, the polar solvent is selected from the group comprising acetonitrile and isopropyl alcohol.

Preferably, during the azeotropic distillation process in step c), the volume of distilled solvent is replenished by successive addition of further portions of solvent.

By using a temperature in the range of 35-55° C. in step a) of the synthesis, a 100% conversion of (2S,3R)-2-hydroxy-3-isobutylsuccinic acid (PKL-018) into (R)-2-((S)-2,2-dimethyl-5-oxo-1,3-dioxolan-4-yl)-4-methylpentanoic acid (PKL-019) was achieved. At a lower temperature, i.e. 20-30° C., despite extending the reaction time to 36 hours, the reaction did not proceed to completion. In turn, too high temperature, i.e. 70° C., resulted in the formation of a number of impurities, including impurities resulting from the condensation of 2,2-dimethoxypropane. Addition of diisopropylethylamine (DIPEA), triethylamine or N-methylmorpholine to the reaction mixture after the reaction was completed to neutralize p-toluenesulfonic acid, pyridinium p-toluenesulfonate or 10-camphorsulfonic acid allowed to obtain the crude product PKL-019 ready for the next step of synthesis without additional purification. Concentration of the reaction mixture without the addition of diisopropylethylamine, resulted in partial decomposition of the product PKL-019 into the substrate PKL-018, because the isopropylidene protecting group is labile in an acidic environment.

Carrying out the reaction in step b) at a temperature from −10° C. to room temperature using diisopropylethylamine (DIPEA) as a base, 2-(1H-benzotriazole-1-yl)-1,1,3,3-tetramethylaminium tetrafluoroborate (TBTU) as a coupling reagent and acetonitrile or methylene chloride as a solvent, crude (R)-N-((S)-3,3-dimethyl-1-methylamino-1-oxobutan-2-yl)-2-((S)-2,2-dimethyl-5-oxo-1,3-dioxolan-4-yl)-4-methylpentanamide (crude PKL-020) was achieved with a yield of 96%-97.1%. Maceration of crude PKL-020 in methyl tert-butyl ether (MTBE) or isopropyl ether at boiling, and then at room temperature led to obtaining (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020) with a purity of 95-99%, diastereoisomer content below 1% and a yield of 61-85%. Moreover, by using the maceration process, purification on a chromatographic column was eliminated. For crude PKL-020 with a purity above 70%, maceration is not required and the next step can be carried out using crude PKL-020. Addition of the tertiary amine in step b) in two portions, the first portion before adding (S)-2-amino-N,3,3-trimethylbutanamide or its hydrochloride, the second portion after adding (S)-2-amino-N,3,3-trimethylbutanamide or its hydrochloride, minimizes the diastereoisomer PKL-020dias formation.

In step c) acetone was added to the mixture after the reaction was completed. Acetone formed with the hydroxylamine residue a less toxic hydroxylamine oxime, which limited potential exposure to genotoxic hydroxylamine during processing of the reaction. Moreover, after the reaction was completed, the remaining water was removed by azeotropic distillation to obtain crystalline marimastat with a chemical and enantiomeric purity of over 99% and a yield of 60-85% starting from PKL-020 (a yield of the entire process starting from PKL-018 is 61-72%). Carrying out the process without the PKL-020 maceration stage allows obtaining crystalline marimastat with a chemical and enantiomeric purity above 99%, with a yield of the entire process starting from PKL-018 of 70-78%. Marimastat obtained by such a process no longer requires an additional purification step, and the amount of individual impurities, including process, degradation, diastereoisomer and enantiomer, is below 0.1%.

The developed process of preparing marimastat according to the invention is repeatable and scalable, which made it possible to transfer it to GMP standard production and synthesize it on a scale of several kilograms. The process does not require chromatographic columns, and the final product is crystalline and meets the requirements of analytical specifications (chemical purity, enantiomeric purity, impurity content, residual solvent content, water content, hydroxylamine content, NMR and IR confirming the structure). The total yield of the entire synthesis process is 61-72% for the process including PKL-020 maceration, and 70-78% for the process without PKL-020 maceration. Moreover, the reagents used for the synthesis, e.g. TBTU and hydroxylamine, are cheaper reagents than their potential substitutes (CDI, EDC, and benzyl derivative of hydroxylamine), which significantly reduces the cost of the whole process. The use of a 50% solution of hydroxylamine in the process, instead of its benzyl derivative, shortens the synthesis by one step of deprotection of the benzyl group, which requires to be carried out under conditions of catalytic hydrogenation, which is dangerous on a large scale and requires suitable reactors and rooms adapted to work with hydrogen.

Another subject of the invention is marimastat prepared by the process according to the invention.

Another subject of the invention is a pharmaceutical composition characterized in that it comprises marimastat according to the invention and a pharmaceutically acceptable carrier. The pharmaceutical composition according to the invention is administered by a route suitable for the type of drug form: enteral (e.g. orally, sublingually, rectally), parenteral (e.g. intravenously, intra-arterially, intramuscularly, subcutaneously, by inhalation), or topically (e.g. intraocularly, epidermally, vaginally).

The pharmaceutical composition according to the invention can be given a variety of pharmaceutical forms, well known to those skilled in the art, e.g. from Remington's Pharmaceutical Sciences, ed. 18, Mack Publ.Co.

Preferably, the pharmaceutical composition is in the form of a tablet, modified-release tablet, pill, capsule, powder, granules, pellets, suspension, emulsion, solution, oral liquid forms, e.g. syrup, solution or suspension for injection, solution for infusion, eye drops, ointment, gel, suppository, globule or therapeutic system, e.g. implant, vaginal ring, nanofiber.

Pharmaceutical forms for oral administration include, for example, tablets, pills, powders, granules, pellets, or capsules comprising solid pharmaceutically acceptable carriers such as corn starch, lactose, sucrose, sorbitol, talc, stearic acid, magnesium stearate, dicalcium phosphate or gums. Tablets or granules may be coated or otherwise processed to provide a dosage unit that provides the advantage of prolonged action.

A variety of substances can be used to form such protective or coating layers, including a variety of polymeric acids and mixtures of polymeric acids with substances such as shellac, ethyl alcohol or cellulose acetate.

Pharmaceutical forms for injection and infusion include sterile aqueous, aqueous-organic and non-aqueous solutions, suspensions, dry substances and tablets for solution or implantation. To prepare the suspension, excipients are used to ensure uniform dispersion of the medicinal substance in the liquid phase, such as polysorbates, lecithin, copolymers of polyoxyethylene with polyoxypropylene, peptizers such as phosphates, polyphosphates and citrates, water-soluble polymers such as carboxymethylcellulose, methylcellulose, polyvinylpyrrolidone, gums or gelatin. Preparations for injection may comprise pharmaceutically acceptable excipients such as pH adjusting and buffering agents, tonicifiers and preservatives. Dry substances are intended for preparing a solution or suspension ex tempore, by dilution with a suitable solvent.

Another subject of the invention is the pharmaceutical composition according to the invention for use as a medicament.

Another subject of the invention is the pharmaceutical composition according to the invention for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

Another subject of the invention is marimastat according to the invention for use as a medicament.

Another subject of the invention is marimastat according to the invention for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

The choice of drug dose and dosage regimen depends on the type of disease, age, weight and health of the patient and can be determined by a specialist based on known treatment regimens. In the treatment of post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complication such as diabetic retinopathy and diabetic foot, the appropriate dose of marimastat according to the invention may be from 0.05 to 50 mg/kg per day, preferably from 0.1 to 10 mg/kg per day. The appropriate dose may be administered to the patient once or several times a day, alone or in combination with other medicinal substances. Such substances can be administered simultaneously in the form of one preparation or separate preparations, or one after the other in an order and time interval determined by a specialist.

### BRIEF DESCRIPTION OF THE DRAWINGS

The subject of the invention is presented in more detail in the examples of implementation and in the drawing, in which:

FIG. 1 shows the structural formula of marimastat (PKL-021);

FIG. 2 shows the ¹H NMR spectrum of PKL-021;

FIG. 3 shows the ¹³C NMR spectrum of PKL-021;

FIG. 4 shows the IR spectrum (KBr tablet) of PKL-021;

FIG. 5 shows the HPLC chromatograms of crude PKL-020, PKL-020 (after maceration) and the diastereoisomer PKL-020dias standard;

FIG. 6 shows the HPLC chromatogram of PKL-021.

### DETAILED DESCRIPTION OF THE INVENTION

### Example 1

### Preparation of (2S,3R)-2-hydroxy-3-isobutylsuccinic acid (PKL-018)

### Step 1: Preparation of diethyl (2R,3R)-2-acetoxy-3-bromosuccinate (PKL-014)

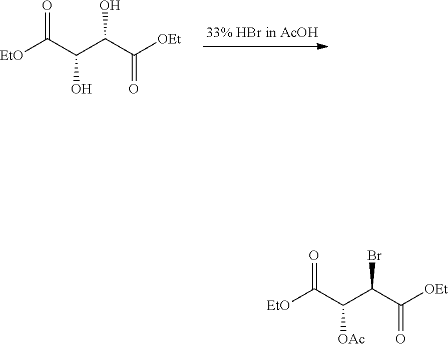 [SMILES: CCOC(=O)[C@@H](O)[C@H](O)C(=O)OCC ; CCOC(=O)[C@@H](OC(C)=O)[C@@H](Br)C(=O)OCC]

Diethyl (−)-D-tartrate (PKL-013, 60.0 g, 291.0 mmol) was cooled to 2° C. Then, not exceeding 5° C., 33% HBr in acetic acid (250.0 mL) was added dropwise. The cooling bath was removed and stirred for 4.5 hours. It was then poured into 600 mL of water with ice and extracted with diethyl ether (400+400+200 mL). The combined organic layers were washed with water (3×100 mL) and saturated sodium chloride solution (100 mL). Dried over Na₂SO₄. The solvent was evaporated to give 94.0 g of a colorless oil. The crude product was used for the next step.

### Step 2: Preparation of diethyl (2R,3R)-2-bromo-3-hydroxysuccinate (PKL-015)

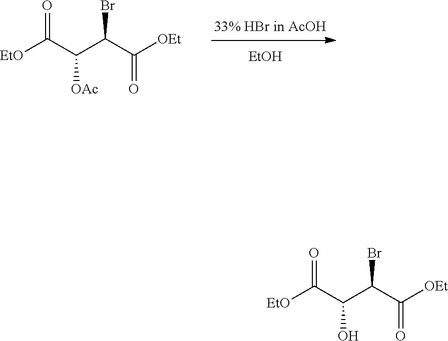 [SMILES: "EtOH" ; CCOC(=O)[C@@H](OC(C)=O)[C@@H](Br)C(=O)OCC ; CCOC(=O)[C@@H](O)[C@@H](Br)C(=O)OCC]

The crude acetylbromohydrin (PKL-014, 94.0 g) was dissolved in anhydrous ethanol (940 mL). Then 33% HBr in acetic acid (31 mL) was added and stirred at boiling while slowly distilling off the solvent. 800 mL was distilled (5 h—TLC—disappearance of the starting material). After cooling to room temperature, diethyl ether (250 mL) and water (250 mL) were added, and layers were separated. The aqueous layer was extracted with diethyl ether (2×250 mL). The combined organic layers were washed with water (2×100 mL), a saturated solution of NaHCO₃ (2×100 mL) and brine (100 mL), and dried over Na₂SO₄. The solvent was evaporated to give 67.0 g of a colorless oil. The crude product was used for the next step.

### Step 3: Preparation of diethyl (2S,3S)-oxirane-2,3-dicarboxylate (PKL-016)

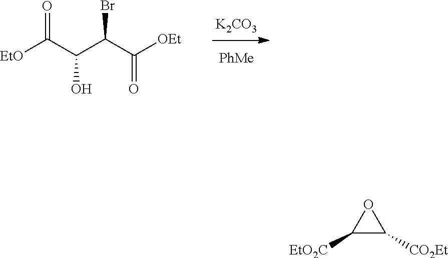

Bromohydrin (PKL-015, 67.0 g, 249 mmol) was dissolved in toluene (670 mL). Then, ground K₂CO₃ (138 g, 996 mmol, 4 eq) was added—the temperature rose to 34° C. The suspension was vigorously stirred on a mechanical stirrer for 2 hours (TLC—complete disappearance of the starting material). The mixture was filtered on a Schott funnel and K₂CO₃ was washed with toluene. The filtrate was washed with water (5-6×100 mL), further with brine (100 mL), and dried over Na₂SO₄. The solvent was evaporated to give 34.9 g of yellow oil (yield: 63%—three steps).

¹H NMR (400 MHz, CDCl₃) δ 4,26 (m, 4H), 3,66 (t, J=0.70 Hz, 2H), 1,31 (m, 6H);

¹³C NMR (100 Hz, CDCl₃) δ 166.8, 62.2, 52.0, 14.0.

### Step 4: Preparation of diethyl (2S,3R)-2-hydroxy-3-isobutylsuccinate (PKL-017)

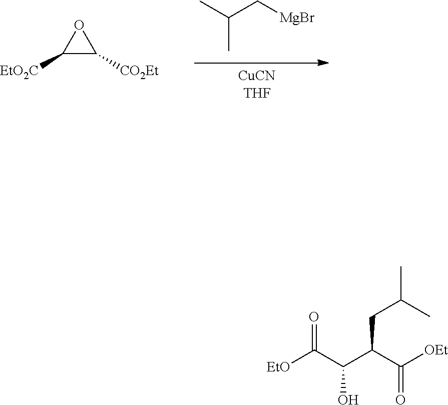

In a reaction vessel with a mechanical stirrer under Ar atmosphere, CuCN (9.5 g, 106.2 mmol) was suspended in anhydrous THF (115 mL). Cooled to −15° C. and previously prepared 1 M Grignard reagent—isobutylmagnesium bromide in THF (101 mL; 101 mmol) was added—temperature rose to 0° C. The mixture was stirred for 20 minutes at a temperature of −5° C., and then not exceeding 25° C., a solution of PKL-016 (10.0 g, 53.1 mmol) in anhydrous THF (50 mL) was added dropwise. The bath was removed and stirred for 1.5 hours (TLC—complete disappearance of the starting material). The reaction was quenched with a saturated ammonium chloride solution (170 mL)—the temperature rose from 20 to 27° C.—stirred for 1.5 hours. It was filtered through Celite. The layers were separated, and the aqueous layer was extracted with diethyl ether (2×50 mL). The combined organic layers were washed with a saturated sodium chloride solution (100 mL), and dried over Na₂SO₄. The solvent was evaporated to give 11.3 g of yellowish oil (yield: 90%). The crude product was used for the next step without purification. ¹H NMR (400 MHz, CDCl₃) δ 4.24 (m, 3H), 4.13 (qd, J=7.15 Hz, J2=1.46 Hz, 2H), 3.19 (dd, J1=7.78 Hz, J2=1.44 Hz, 1H), 2.92 (ddd, J1=8.34 Hz, J2=6.79 Hz, J3=3.66 Hz, 1H), 1.74 (m, 1H), 1.66 (m, 1H), 1.49 (ddd, J1=12.10 Hz, J2=7.67 Hz, J3=6.19 Hz, 1H), 1.29 (t, J=7.15 Hz, 3H), 1.23 (t, J=7.14 Hz, 3H), 0.92 (ddd, J1=6.22 Hz, J2=4.59 Hz, J3=1.48 Hz, 6H); ¹³C NMR (100 Hz, CDCl₃) δ 173.4, 173.0, 71.3, 61.7, 60.8, 46.5, 36.9, 25.7, 22.4, 22.3, 14.13, 14.10.

### Step 5: Preparation of (2S,3R)-2-hydroxy-3-isobutylsuccinic acid (PKL-018)

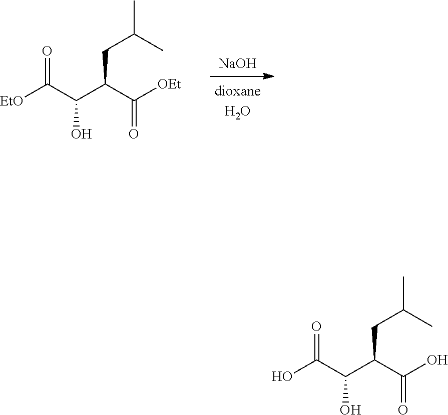

The crude hydroxyester (PKL-017, 11.3 g, 45.9 mmol) was dissolved in 1,4-dioxane (68 mL). Then, sodium hydroxide solution (7.3 g, 183.2 mmol) in water (45 mL) was added. The reaction was stirred at room temperature for 21 h (TLC—complete disappearance of the starting material). It was extracted with diethyl ether (2×20 mL). The combined organic layers were washed with water (20 mL) and discarded. The combined aqueous layers were acidified with 6M HCl to pH 1. The water was evaporated and the residue was evaporated with acetonitrile. Acetonitrile was added again and filtered through Celite. The acetonitrile was evaporated to give 8.66 g of orange oil (yield: 85.7%—two steps).

¹H NMR (400 MHz, CDCl₃) δ 4.33 (t, J=1.82 Hz, 1H), 3.09 (m, 1H), 1.72 (m, 3H), 0.94 (t, J=3.60 Hz, 6H);

¹³C NMR (100 Hz, CDCl₃) δ 178.9, 178.0, 70.4, 46.2, 36.6, 25.5, 22.5, 22.1;

LC-MS [M−H]⁻=188.8; HPLC: 83.9%.

The obtained PKL-018 was used to prepare marimastat (PKL-021).

### Example 2

### Preparation of (S)-2-amino-N,3,3-trimethylbutanamide hydrochloride (PKL-012)

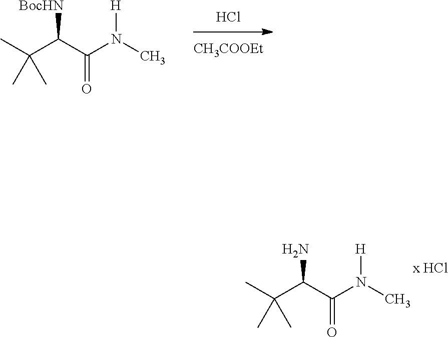

A solution of t-butyloxycarboamide (PKL-011, 335 g, 1.44 mol) in ethyl acetate (800 mL) was added dropwise to a suspension of PKL-012 sample (8.2 g, previously obtained) in 2M HCl in ethyl acetate (1750 mL, 3.5 mol HCl) at room temperature for 4.5 hours. Formation of white precipitate was observed. Then, the mixture was stirred for additional 22 hours at room temperature to complete crystallization. The crystals were filtered off, washed with ethyl acetate, and dried on air to obtain 251.2 g (243 g after subtraction of 8.2 g of PKL-012, initially added to facilitate the crystallization, 93% yield) of pale yellow crystalline material.

¹H NMR (400 MHz, D₂O) δ 3.68 (s, 1H), 2.80 (s, 3H), 1.07 (s, 9H);

¹³C NMR (100 Hz, D₂O) δ 168.9, 62.0, 32.7, 25.7, 25.6;

LC-MS [M+H]⁺=145.1; HPLC: 99.7%.

The obtained PKL-012 was used to prepare marimastat (PKL-021).

### Example 3

### Preparation of marimastat (PKL-021)

An exemplary process of preparing PKL-021 is shown in Scheme 5.

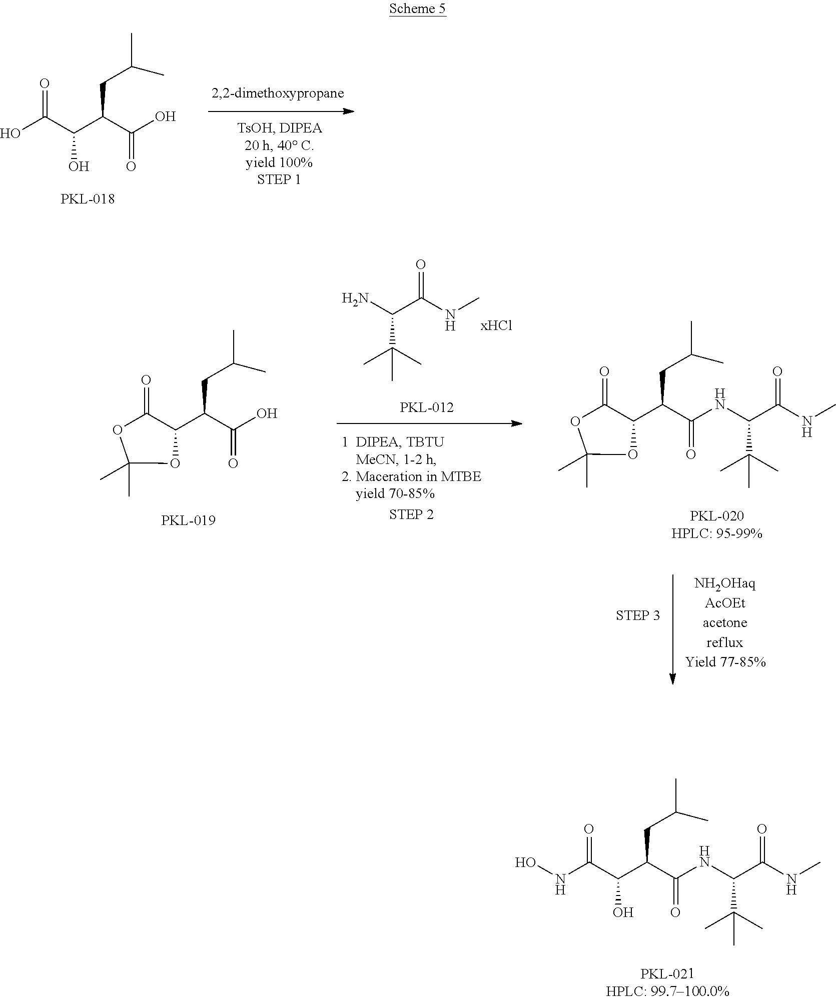

PKL-021 and intermediate products: PKL-019 and PKL-020 were characterized by the following methods: NMR, MS, LC-MS, IR, elemental analysis, and the purity was determined using HPLC. ¹H and ¹³C NMR measurements were performed on a Varian 400 MHz spectrometer. LC-MS analyses were performed on a Shimadzu 2020 chromatograph using the electrospray ionization method.

Compound PKL-021 was further characterized by two-dimensional NMR, IR and elemental analysis. Two-dimensional COSY, HSQC spectra were performed in DMSO-d₆ at room temperature on a Varian 400 MHz spectrometer. IR analysis was performed on a Nicolet iS10 spectrometer in the range of 4000-400 cm⁻¹, with a resolution of 4 cm⁻¹. The PKL-021 sample was prepared in a KBr tablet, approximately 1.5 mg of the compound/200 mg of KBr.

The purity of PKL-019, PKL-020 and PKL-021 was tested by HPLC on a Shimadzu 3050C 3D chromatograph.

### Step 1: Preparation of (R)-2-((S)-2,2-dimethyl-5-oxo-1,3-dioxolan-4-yl)-4-methylpentanoic acid (PKL-019)

p-Toluenesulfonic acid (p-TsOH, 18 g, 105 mmol, 0.05 eq) was added to a solution of PKL-018 (465 g of crude PKL-018, 2.05 mol) in 2,2-dimethoxypropane (5 L). The reaction mixture was stirred at 40° C. for 24 hours. The progress of the reaction was monitored by HPLC and TLC (TLC:CHCl₃:CH₃OH:H₂O 6:4:0.3). After the reaction was completed (HPLC:PKL-018≤3%), diisopropylethylamine (DIPEA, 46 mL, 0.1 eq) was added, and the mixture was concentrated to give a brown oil. The crude product PKL-019 was used in the next step without additional purification.

Reaction yield: 100%.

¹H NMR (400 MHz, CDCl₃) δ 4.45 (1H, d, J=4.9 Hz), 4.11 (1H, q, J=7.1 Hz), 3.0 (1H, ddd, J=8.3 Hz, J=6.3 Hz, 4.8 Hz), 2.02 (1H, s), 1.80-1.75 (2H, m), 1.58 (3H, s), 1.52 (3H, s), 0.95 (3H, d, J=2.2 Hz), 0.93 (3H, J=2.0 Hz);

LC-MS [M−H]−=228.8; HPLC: 77.24%.

Other tertiary amines such as triethylamine (Et₃N) and N-methylmorpholine (NMM) were also tested and a similar result was obtained as with DIPEA. It is preferable to use the same amine that is used in the next step, i.e. coupling PKL-019 with PKL-012.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

The crude PKL-019 (2.05 mol) was dissolved in acetonitrile (5 L). Then, 2-(1H-benzotriazole-1-yl)-1,1,3,3-tetramethylaminium tetrafluoroborate (TBTU) (929 g, 2.89 mol, 1.4 eq) and diisopropylethylamine (DIPEA, 1.38 L; 7.89 mol, 3.8 eq) were added to the clear solution. Finally, PKL-012 (475 g, 2.63 mol, 1.3 eq) was added portionwise, and the reaction mixture was stirred at room temperature. The progress of the reaction was monitored by TLC method (10% CH₃OH/CH₂Cl₂). The reaction was usually completed within 2 hours. The mixture was diluted with ethyl acetate (5 L) and washed with 5% KHSO₄ (3×5 L), a saturated solution of NaHCO₃ (3×5 L), and brine (2.5 L). The organic layer was dried over anhydrous Na₂SO₄. The solvent was evaporated to give 700 g of brown oil (yield of the crude PKL-020:96%).

The crude product was macerated at boiling in methyl tert-butyl ether (MTBE) (5 L) for 60 min. The mixture was cooled to room temperature and stirred for 17 hours. The crystals were filtered off, washed with MTBE (3×1.0 L), and dried at room temperature to constant weight to give a white solid of PKL-020 (577 g, 79% yield).

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 98.7%.

Alternatively, the preparation of PKL-020 was performed in methylene chloride as described below.

The crude PKL-019 (5.26 mmol) was dissolved in methylene chloride (10 mL). Then, 2-(1H-benzotriazole-1-yl)-1,1,3,3-tetramethylaminium tetrafluoroborate (TBTU, 1.86 g, 5.78 mmol) and diisopropylethylamine (DIPEA, 2.7 mL, 15.78 mol) were added to the clear solution. Finally, PKL-012 (950 mg, 5.26 mol) was added portionwise and the reaction mixture was stirred at room temperature. The progress of the reaction was monitored by TLC method (10% CH₃OH/CH₂Cl₂). The reaction was usually completed within 2 hours. The reaction mixture was washed with 5% KHSO₄ (3×10 mL), saturated solution of NaHCO₃ (3×10 mL) and brine (5 mL). The organic layer was dried over anhydrous Na₂SO₄. After evaporating the solvent, 1.81 g of a brown oil was obtained (the crude PKL-020 yield: 97.1%).

The crude product was macerated at boiling in methyl tert-butyl ether (MTBE, 10 mL) for 60 min. The mixture was cooled to room temperature and stirred for 17 hours. The crystals were filtered off, washed with MTBE (3×2 mL) and dried at room temperature to constant weight, obtaining 1.49 g of a white solid of PKL-020 (80% yield).

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 98.5%.

In the course of the work, it was observed that, depending on the reaction conditions used, i.e. the type of coupling reagent, the tertiary amine used, the method of its addition and the type of solvent, in addition to the desired product PKL-020, the diasteroisomer PKL-020dias shown below is also formed.

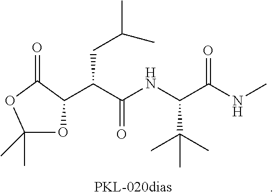

A number of different conditions were tested, the results of which are presented in Table 1. Conducting the reaction to obtain PKL-020 at room temperature, using diisopropylethylamine (DIPEA) as a base, 2-(1H-benzotriazole-1-yl)-1,1,3,3-tetramethylaminium tetrafluoroborate (TBTU) as a coupling reagent and acetonitrile (ACN) as a solvent, the reaction yield was 96%, and the purity of the obtained crude PKL-020 was 85% with the content of PKL-020dias. 4.4% (item 1, Table 1).

Good results were also obtained using methylene chloride (DCM) as a solvent in the presence of DIPEA and using TBTU as a coupling reagent (item 2, Table 1). Replacing acetonitrile with methylene chloride resulted in a decrease in the amount of diastereoisomer formed to 1.7%. Two subsequent experiments, i.e. items 3 and 4 in Table 1, prove the beneficial effect of methylene chloride used as a solvent on the ratio of the product PKL-020 to PKL-020dias. The amount of PKL-020dias. formed in both samples was 8.5% vs. 4.9%. By far the weakest result was obtained when triethylamine (Et₃N) was used as a tertiary amine (item 5, Table 1). In this case, the amount of PKL-020dias. formed increased to 32.0%.

In turn, tests carried out in which 1,1-carbonyldiimidazole (CDI) was used as a coupling reagent showed that the reaction did not proceed at all or the amount of PKL-020dias. formed was above 9% (items 6-8, Table 1).

TABLE 1

| | | | | | Crude | |
|---|---|---|---|---|---|---|
| | | | Coupling | Yield | PKL-020 | PKL-020dias. |
| No. | Amine | Solvent | reagent | (crude PKL-020) | (HPLC) | (HPLC) |
| 1 | DIPEA | ACN | TBTU | 96% | 85% | 4.4% |
| 2 | DIPEA | DCM | TBTU | 97.1% | 76.5% | 1.7% |
| 3 | NMM | ACN | TBTU | 86.9% | 68.5% | 8.5% |
| 4 | NMM | DCM | TBTU | 102.9% | 73.3% | 4.9% |
| 5 | Et₃N | ACN | TBTU | 88.0% | 47.9% | 32.0% |
| 6* | DIPEA | ACN | CDI | — | — | — |
| 7 | NMM | DCM | CDI | 70.6% | 56.1% | 9.4% |
| 8 | NMM | ACN | CDI | 69.0% | 57.5% | 9.7% |
| 9 | DIPEA | DMF | TBTU | 52.0% | 79.1% | 4.75% |

*no reaction product

In addition, the method of adding the tertiary amine influences the amount of diastereoisomer. Experiments were performed using DIPEA, and it was shown that the active ester formed from PKL-019 and TBTU was easily epimerized in an alkaline environment (item 1, 2, Table 2). According to the above, the lowest amount of diastereoisomer is formed when DIPEA is added in two portions: the first portion of the tertiary amine (1-1.5 eq) is added before the addition of PKL-012 and is used to generate the active ester, the second portion (2-2.8 eq) is added after adding PKL-012, it releases PKL-012 hydrochloride and initiates the amidation reaction (item 3, Table 2).

TABLE 2

| | Method of adding reagents to the solution | PKL-020:PKL-020dia |
|---|---|---|
| No. | of PKL-019 in MeCN | (HPLC) |
| 1 | TBTU, DIPEA (3 eq), stirring for 1 h, | 83:17 |
| | then adding PKL-012 | |
| 2 | TBTU, DIPEA (3 eq), stirring for 3 h, | 53:47 |
| | then adding PKL-012 | |
| 3 | TBTU, DIPEA (1 eq), stirring for 0,5 h, | 98:2 |
| | then adding PKL-012, DIPEA (2 eq) | |

As a result of the coupling reaction carried out in acetonitrile or methylene chloride, in the presence of DIPEA as a base and TBTU as a coupling reagent, the crude product PKL-020 was obtained, which was isolated by subjecting the reaction mixture to an extraction process. Then, the crude PKL-020 obtained in the above manner was macerated at boiling in methyl tert-butyl ether (MTBE) or isopropyl ether, and then at room temperature. PKL-020 was obtained with a purity of 95-99%, PKL-020dias content below 1% and a yield of 79-85%. Using the maceration process, purification on a chromatographic column was eliminated. Exemplary chromatograms of crude PKL-020, PKL-020 (after maceration) and the diastereoisomer PKL-020dias standard are shown in FIG. 5. HPLC analyzes were performed on a Shimadzu apparatus, using a ReproSil-Pur Basic C8 column, 3 μm, 50×3 mm, UV detector (DAD)/205 nm, samples were dissolved in acetonitrile, a mixture of H₂O:MeOH:ACN:HCOOH was used as a mobile phase.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

PKL-020 (560 g, 1.57 mol, 1 eq) was dissolved in ethyl acetate (AcOEt, 5.6 L) at 50° C. and NH₂OH (370 mL, 50% solution in water, 5.97 mol, 3.8 eq) was added. The suspension was stirred at boiling for 1 h (TLC monitoring: 10% CH₃OH/CH₂Cl₂). Then, the temperature of the reaction mixture was decreased to 45° C., and 1.1 L of acetone was added dropwise. The reaction was stirred at boiling for additional 30 minutes. Then, the mixture was subjected to azeotropic distillation from the mixture of AcOEt and acetone to remove water. The volume of distilled solvent was replenished by successive addition of further portions of AcOEt. Then, the reaction mixture was cooled to room temperature and stirred for 17 hours. The crystals were filtered off, washed with AcOEt (2×1.0 L), acetone (1.0 L), and dried to obtain 445 g of PKL-021 (77% yield) as a white solid.

LC-MS [M−H]⁻=329.95; HPLC: 100%; elemental analysis for C₁₅H₂₉N₃O₅, calculated: C— 54.36%, H— 8.82%, N— 12.68%, measured: C—54.36%, H—8.94%, N—12.66%.

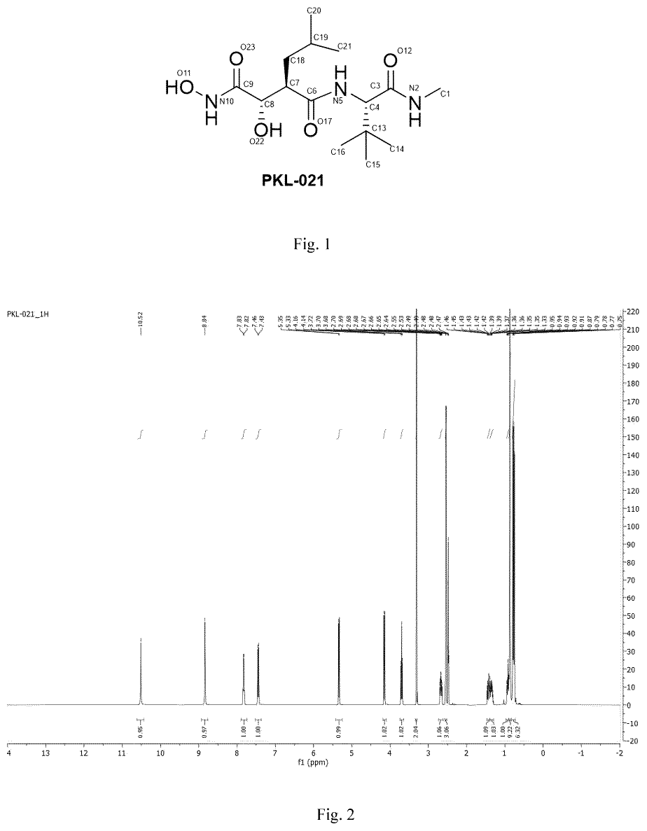

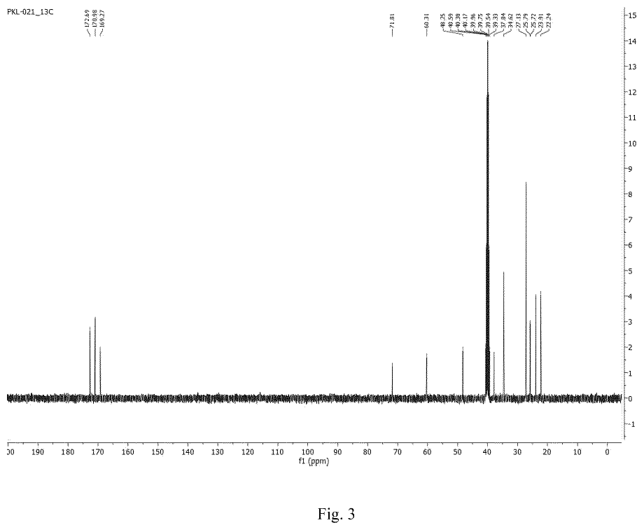

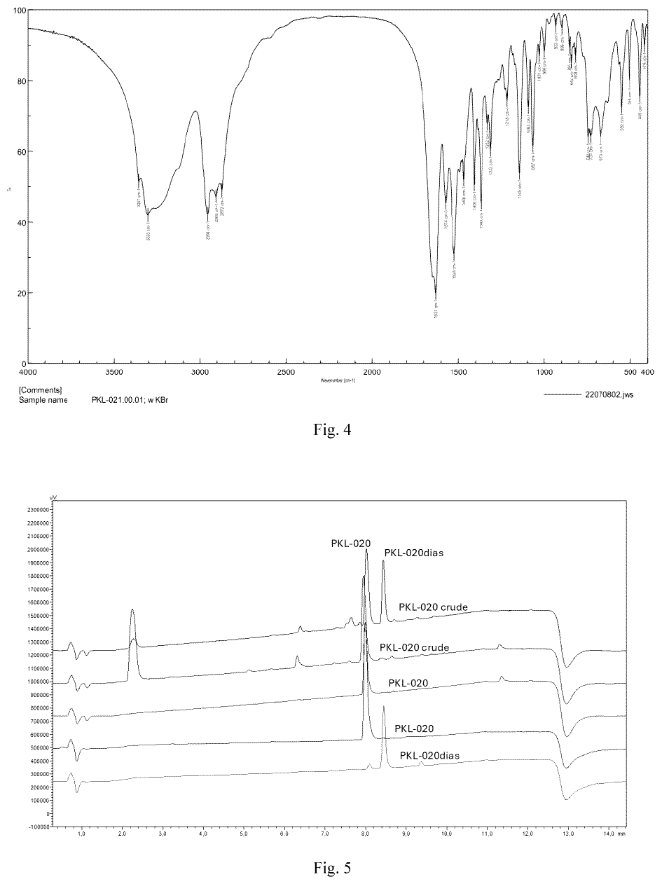

The results of the NMR analysis along with signal assignments, the IR spectrum for compound PKL-021 are presented in FIGS. 2, 3 and 4 and in Tables 3 and 4.

TABLE 3
¹H and ¹³C NMR chemical shifts
of compound PKL-021 in DMSO-d₆.

| | ¹³C-NMR | ¹H-NMR | Description of ¹H- |
|---|---|---|---|
| Number | [ppm] | [ppm] | NMR signals |
| C3; C6; C9 | 172.7; 171.0; 169.3 | — | — |
| C8 | 71.8 | 3.70 | t, J = 8.00 Hz, 1H |
| C4 | 60.3 | 4.15 | d, J = 9.51 Hz, 1H |
| C7 | 48.33 | 2.67 | ddd, J1 = 10.59 Hz, |
| | | | J2 = 8.19 Hz, |
| | | | J3 = 4.09 Hz, 1H |
| C18 | 37.80 | 1.43 | m, 1H |
| | | 0.93 | m, 1H |
| C13 | 34.6 | — | — |
| C14; C15; C16 | 27.1 | 0.87 | s, 9H |
| C19 | 25.8 | 1.34 | m, 1H |
| C1 | 25.7 | 2.54 | d, J = 4.58 Hz, 3H |
| C20; C21 | 23.9; 22.2 | 0.77 | dd, J1 = 9.79 Hz, J2 = |
| | | | 6.47 Hz, 6H |
| N10 | — | 10.52 | s, 1H |
| O11 | — | 8.84 | s, 1H |
| N2 | — | 7.83 | q, J = 4.66 Hz, 1H |
| N5 | — | 7.45 | d, J = 9.54 Hz, 1H |
| O22 | — | 5.34 | d, J = 7.85 Hz, 1H |

TABLE 4
Characteristic absorption bands of the
IR spectrum of compound PKL-021.

| bands [cm⁻¹] | Description |
|---|---|
| 3400-3100 | Stretching vibrations of O—H bonds and |
| | stretching vibrations of N—H bonds |
| 2956, 2906 and 2872 | Stretching vibrations of C—H alkyl bonds |
| 1631 | Stretching vibrations of C═O bonds of amide |
| | groups |
| 1574 and 1524 | Bending vibrations of N—H bonds |
| 1469 | Bending vibrations of C—H alkyl bonds |
| 1408 | Bending vibrations of O—H bonds |
| 1368 | Bending vibrations of C—H bonds in methyl |
| | groups |
| 1312, 1218 and 1145 | Stretching vibrations of C—N bonds |
| 1067 | Stretching vibrations of C—O bonds |
| 842-673 | Bending vibrations of O—H bonds, bending |
| | vibrations of N—H bonds, stretching |
| | vibrations of N—O bonds and bending |
| | vibrations of C—H bonds |

Alternatively, the reaction of PKL-020 with hydroxylamine was also carried out in other solvents, i.e. other lower carboxylic acid esters, e.g. isopropyl acetate, non-polar solvents, e.g. 2-methyltetrahydrofuran, 1,2-dimethoxyethane, methylene chloride, chloroform and polar solvents, e.g. acetonitrile, isopropyl alcohol. The results of the reactions carried out to obtain marimastat (PKL-021) in Step 3 are presented in Table 5. Using the above solvents, PKL-021 was obtained with a yield of over 60% and a purity of over 99%. It is particularly preferred to use ethyl acetate, methylene chloride, 2-methyltetrahydrofuran or chloroform as a solvent in Step 3. Using ethyl acetate, methylene chloride, 2-methyltetrahydrofuran or chloroform, PKL-021 was obtained with a purity of 100%, 99.95%, 99.95% and 99.93%, respectively, and a yield of over 80%.

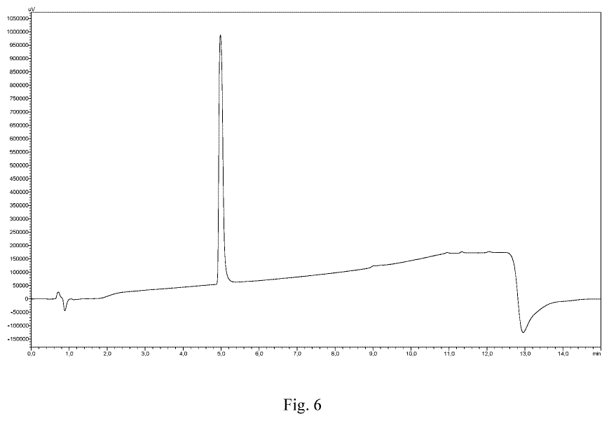

The obtained marimastat (PKL-021) did not require an additional purification step, and the amount of individual impurities, including process, degradation, diastereoisomer and enantiomer, was below 0.1%. The exemplary HPLC chromatogram of PKL-021 is shown in FIG. 6.

TABLE 5

| No. | solvent | purity | yield |
|---|---|---|---|
| 1 | ethyl acetate | 100% | 77-85% |
| 2 | isopropyl acetate | 99.45% | 80% |
| 3 | 1,2-dimethoxyethane | 99.70% | 72% |
| 4 | acetonitrile | 99.28% | 64% |
| 5 | methylene chloride | 99.95% | 83% |
| 6 | 2-methyltetrahydrofuran | 99.95% | 85% |
| 7 | isopropanol | 99.54% | 75% |
| 8 | chloroform | 99.93% | 80% |

### Example 4

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Step 1 is the same as in Example 3.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

The crude PKL-019 (44.7 mmol) was dissolved in acetonitrile (100 mL) and the resulting solution was cooled to 0-5° C. Then, 2-(1H-benzotriazole-1-yl)-1,1,3,3-tetramethylaminium tetrafluoroborate (TBTU) (18.6 g; 57.9 mmol; 1.3 eq) and diisopropylethylamine (DIPEA) (9.2 mL; 52.6 mmol, 1.2 eq) were added to the clear solution. Then, PKL-012 (9.5 g; 52.6 mmol; 1.2 eq) and diisopropylethylamine (DIPEA) (18.3 mL; 105.2 mmol, 2.4 eq) were added portionwise. The reaction mixture was stirred at a temperature of 0-5° C. The progress of the reaction was monitored by TLC method (10% CH₃OH/CH₂Cl₂). The reaction was usually completed within 1 hour. The mixture was diluted with ethyl acetate (100 mL) and washed with 5% KHSO₄ (3×100 mL), a saturated solution of NaHCO₃ (3×100 mL), and brine (50 mL). The organic layer was dried over anhydrous Na₂SO₄. The purity of the crude PKL-020 was 77%. After filtering off the drying agent, the residue was used for the next step.

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 80.5%.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

The PKL-020 solution (44.7 mmol in AcOEt) was diluted to a total volume of 200 mL with ethyl acetate and heated to 45° C. Then, NH₂OH (12.2 mL, 50% solution in water, 200 mmol, 4.5 eq) was added. The suspension was stirred at a temperature of 45° C. for 1 h (TLC monitoring: 10% CH₃OH/CH₂Cl₂). Then, 30 mL of acetone were added dropwise. The reaction mixture was stirred at boiling for additional 30 minutes. Then, the reaction mixture was subjected to azeotropic distillation from the mixture of AcOEt and acetone to remove water. The volume of distilled solvent was replenished by successive addition of further portions of AcOEt. Then, the reaction mixture was cooled to room temperature and stirred for 17 hours. The crystals were filtered off, washed with AcOEt (2×10 mL), acetone (10 mL), and dried to obtain 11.5 g of PKL-021 (78% yield) as a white solid.

¹H and ¹³C NMR chemical shifts of compound PKL-021 in DMSO-d₆ are presented in Table 3. Characteristic absorption bands of the IR spectrum of compound PKL-021 are presented in Table 4.

### Example 5

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Step 1 is the same as in Example 3.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

The crude PKL-019 (15.8 mmol) was dissolved in acetonitrile (30 mL) and cooled to −10° C. Then, 2-(1H-benzotriazole-1-yl)-1,1,3,3-tetramethylaminium tetrafluoroborate (TBTU) (5.56 g; 17.35 mmol; 1.1 eq) and diisopropylethylamine (DIPEA) (2.75 mL; 15.76 mmol, 1.0 eq) were added to the clear solution. Then, PKL-012 (2.85 g; 15.77 mmol; 1.0 eq) and diisopropylethylamine (DIPEA) (5.51 mL; 31.54 mmol, 2.0 eq) were added portionwise. The reaction mixture was stirred at −10° C. for 1 hour. The progress of the reaction was monitored by TLC method (10% CH₃OH/CH₂Cl₂). The mixture was diluted with ethyl acetate (30 mL) and washed with 5% KHSO₄ (3×30 mL), a saturated solution of NaHCO₃ (3×30 mL), and brine (15 mL). The organic layer was dried over anhydrous Na₂SO₄. The purity of the crude PKL-020 was 82% (84% yield). The crude product was macerated at boiling in methyl tert-butyl ether (MTBE) (30 mL) for 60 min. The mixture was cooled to room temperature and stirred for 17 hours. The crystals were filtered off, washed with MTBE (3×10 mL), and dried at room temperature to constant weight to give a white solid of PKL-020 (3.4 g, 61% yield).

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 97.1%.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 6

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Step 1 is the same as in Example 3.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

The crude PKL-019 (15.8 mmol) was dissolved in acetonitrile (30 mL). Then, 2-(1H-benzotriazole-1-yl)-1,1,3,3-tetramethylaminium tetrafluoroborate (TBTU) (5.56 g; 17.35 mmol; 1.1 eq) and 4-methylmorpholine (NMM) (5.2 mL; 47.3 mmol, 3.0 eq) were added to the clear solution. Finally, PKL-012 (475 g; 2.63 mol; 1.3 eq) was added portionwise and the reaction mixture was stirred at room temperature. The progress of the reaction was monitored by TLC method (10% CH₃OH/CH₂Cl₂). The reaction was usually completed within 2 hours. The mixture was diluted with ethyl acetate (30 mL) and washed with 5% KHSO₄ (3×30 mL), a saturated solution of NaHCO₃ (3×30 mL), and brine (15 mL). The organic layer was dried over anhydrous Na₂SO₄. The solvent was evaporated to give 4.9 g of brown solid (yield of the crude PKL-020:86.9%). The crude product was macerated at boiling in methyl tert-butyl ether (MTBE) (30 mL) for 60 min. The mixture was cooled to room temperature and stirred for 17 hours. The crystals were filtered off, washed with MTBE (3×10 mL), and dried at room temperature to constant weight to give a white solid of PKL-020 (2.74 g, 49% yield).

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 96.8%.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 7

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Step 1 is the same as in Example 3.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

Step 2 is the same as in Example 3.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

PKL-020 (3 g, 8.42 mmol, 1 eq) was dissolved in 30 mL of methylene chloride at room temperature. Then, NH₂OH (1.98 mL, 50% solution in water, 32 mmol, 3.8 eq) was added. The suspension was stirred at room temperature for 24 h (TLC monitoring: 10% CH₃OH/CH₂Cl₂). Then, 6 mL of acetone was added dropwise. The reaction was stirred at boiling for 30 minutes. Then, the mixture was subjected to azeotropic distillation from the mixture of methylene chloride and acetone to remove water. The volume of distilled solvent was replenished by successive addition of further portions of methylene chloride. Then, the reaction mixture was cooled to room temperature and stirred for 17 hours. The crystals were filtered off, washed with methylene chloride (2×6 mL), acetone (6 mL), and dried to obtain 2.31 g of PKL-021 (83% yield) as a white solid.

¹H and ¹³C NMR chemical shifts of compound PKL-021 in DMSO-d₆ are presented in Table 3. Characteristic absorption bands of the IR spectrum of compound PKL-021 are presented in Table 4.

### Example 8

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Step 1 is the same as in Example 3.

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

The crude PKL-019 (5.26 mmol) was dissolved in acetonitrile (10 mL). Then, 2-(1H-benzotriazole-1-yl)-1,1,3,3-tetramethylaminium tetrafluoroborate (TBTU) (1.86 g; 5.78 mmol; 1.1 eq) and triethylamine (NEt₃) (2.2 mL; 15.78 mmol, 3.0 eq) were added to the clear solution. Finally, PKL-012 (1.2 g; 6.84 mol; 1.3 eq) was added portionwise, and the reaction mixture was stirred at room temperature. The progress of the reaction was monitored by TLC method (10% CH₃OH/CH₂Cl₂). The reaction was usually completed within 2 hours. The mixture was diluted with ethyl acetate (10 mL) and washed with 5% KHSO₄ (3×10 mL), a saturated solution of NaHCO₃ (3×10 mL), and brine (10 mL). The organic layer was dried over anhydrous Na₂SO₄. The solvent was evaporated to give 1.65 g of brown solid (yield of the crude PKL-020:88%). The crude product was macerated at boiling in methyl tert-butyl ether (MTBE) (10 mL) for 60 min. The mixture was cooled to room temperature and stirred for 17 hours. The crystals were filtered off, washed with MTBE (3×3 mL), and dried at room temperature to constant weight to give a white solid of PKL-020 (0.66 g, 35% yield).

¹H NMR (400 MHz, CDCl₃) δ 6.69 (1H, d, J=9.3 Hz), 6.37 (1H, d, J=5.0 Hz), 4.43 (1H, d, J=5.8 Hz), 4.28 (1H, d, J=9.3 Hz), 2.78 (3H, d, J=4.8 Hz), 2.77-2.75 (1H, m), 1.86 (1H, brs), 1.75-1.61 (1H, m), 1.60 (3H, s), 1.50 (3H, s), 1.17 (1H, s), 0.98 (9H, s), 0.90 (6H, t, J=6.1 Hz);

¹³C NMR (100 Hz, CDCl₃) δ 172, 170.8, 170.6, 110.2, 74.6, 60.6, 49.4, 47.3, 36.8, 34.6, 26.9, 26.7, 26.5, 26.0, 25.8, 25.7, 23.0, 21.9;

LC-MS [M+H]⁺=357.2; HPLC: 95.1%.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 9

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

p-Toluenesulfonic acid (p-TsOH) (36 mg, 0.21 mmol) was added to a solution of PKL-018 (1 g, 5.26 mmol) in 2,2-dimethoxypropane (10 mL). The reaction mixture was stirred at a temperature of 40° C. for 20 hours. The progress of the reaction was monitored by HPLC and TLC methods (TLC:CHCl₃:CH₃OH:H₂O 6:4:0.3). After the reaction was completed (HPLC: PKL-018≤3%), N-methylmorpholine (NMM) (58 μL, 0.53 mmol) was added, and the mixture was concentrated to give a brown oil. The crude product PKL-019 was used in the next step without additional purification.

Reaction yield: 100%.

¹H NMR (400 MHz, CDCl₃) δ 4.45 (1H, d, J=4.9 Hz), 4.11 (1H, q, J=7.1 Hz), 3.0 (1H, ddd, J=8.3 Hz, J=6.3 Hz, 4.8 Hz), 2.02 (1H, s), 1.80-1.75 (2H, m), 1.58 (3H, s), 1.52 (3H, s), 0.95 (3H, d, J=2.2 Hz), 0.93 (3H, J=2.0 Hz);

LC-MS [M−H]−=228.8

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

Step 2 is the same as in Example 6.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 10

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

p-Toluenesulfonic acid (p-TsOH) (36 mg, 0.21 mmol) was added to a solution of PKL-018 (1 g, 5.26 mmol) in 2,2-dimethoxypropane (10 mL). The reaction mixture was stirred at a temperature of 40° C. for 20 hours. The progress of the reaction was monitored by HPLC and TLC methods (TLC: CHCl₃:CH₃OH:H₂O 6:4:0.3). After the reaction was completed (HPLC: PKL-018≤3%), triethylamine (NEt₃) (74.5 μL, 0.53 mmol) was added, and the mixture was concentrated to give a brown oil. The crude product PKL-019 was used in the next step without additional purification.

Reaction yield: 100%.

¹H NMR (400 MHz, CDCl₃) δ 4.45 (1H, d, J=4.9 Hz), 4.11 (1H, q, J=7.1 Hz), 3.0 (1H, ddd, J=8.3 Hz, J=6.3 Hz, 4.8 Hz), 2.02 (1H, s), 1.80-1.75 (2H, m), 1.58 (3H, s), 1.52 (3H, s), 0.95 (3H, d, J=2.2 Hz), 0.93 (3H, J=2.0 Hz);

LC-MS [M−H]−=228.8

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

Step 2 is the same as in Example 8.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 11

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

(1S)-(+)-10-camphorsulfonic acid (49 mg, 0.21 mmol) was added to a solution of PKL-018 (1 g, 5.26 mmol) in 2,2-dimethoxypropane (10 mL). The reaction mixture was stirred at a temperature of 40° C. for 20 hours. The progress of the reaction was monitored by HPLC and TLC methods (TLC: CHCl₃:CH₃OH:H₂O 6:4:0.3). After the reaction was completed (HPLC: PKL-018≤3%), diisopropylethylamine (DIPEA) (93 μL, 0.53 mmol) was added, and the mixture was concentrated to give a yellow oil. The crude product PKL-019 was used in the next step without additional purification.

Reaction yield: 100%.

¹H NMR (400 MHz, CDCl₃) δ 4.45 (1H, d, J=4.9 Hz), 4.11 (1H, q, J=7.1 Hz), 3.0 (1H, ddd, J=8.3 Hz, J=6.3 Hz, 4.8 Hz), 2.02 (1H, s), 1.80-1.75 (2H, m), 1.58 (3H, s), 1.52 (3H, s), 0.95 (3H, d, J=2.2 Hz), 0.93 (3H, J=2.0 Hz);

LC-MS [M−H]−=228.8

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

Step 2 is the same as in Example 3.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 12

### Preparation of marimastat (PKL-021)

### Step 1: Preparation of (R)-2-((S)-2,2-dimethyl-5-oxo-1,3-dioxolan-4-yl)-4-methylpentanoic acid (PKL-019)

Pyridinium p-toluenesulfonate (PPTS) (13.2 mg, 0.053 mmol) was added to a solution of PKL-018 (0.1 g, 0.526 mmol) in 2,2-dimethoxypropane (1 mL). The reaction mixture was stirred at a temperature of 40° C. for 20 hours. The progress of the reaction was monitored by HPLC and TLC methods (TLC: CHCl₃:CH₃OH:H₂O 6:4:0.3). After the reaction was completed (HPLC:PKL-018≤3%), diisopropylethylamine (DIPEA) (9.3 μL, 0.053 mmol) was added, and the mixture was concentrated to give a yellow oil. The crude product PKL-019 was used in the next step without additional purification.

Reaction yield: 100%.

¹H NMR (400 MHz, CDCl₃) δ 4.45 (1H, d, J=4.9 Hz), 4.11 (1H, q, J=7.1 Hz), 3.0 (1H, ddd, J=8.3 Hz, J=6.3 Hz, 4.8 Hz), 2.02 (1H, s), 1.80-1.75 (2H, m), 1.58 (3H, s), 1.52 (3H, s), 0.95 (3H, d, J=2.2 Hz), 0.93 (3H, J=2.0 Hz);

LC-MS [M−H]−=228.8

### Step 2: Preparation of (R)-N-((S)-3,3-dimethyl-1-methylamino-1-oxobutan-2-yl)-2-((S)-2,2-dimethyl-5-oxo-1,3-dioxolan-4-yl)-4-methylpentanamide (PKL-020)

Step 2 is the same as in Example 3.

### Step 3: Preparation of (2R,3S)-N-1-((S)-3,3-dimethyl-1-methylamino-1-oxobutan-2-yl)-N-4,3-dihydroxy-2-isobutylsuccinamide (PKL-021)

Step 3 is the same as in Example 3.

### Example 13

### Preparation of Tablets Comprising marimastat (PKL-021)

Tablets comprising PKL-021 were produced by wet granulation according to the following steps:
1. Preparation of granulation liquid
2. Granulation
3. Calibration of granulate before drying
4. Drying
5. Calibration of granulate after drying
6. Mixing the granulate with external phase components
7. Tableting

### Step 1: Preparation of Granulation Liquid

A binder is added to water and stirred until completely dissolved.

### Step 2: Granulation

A mixture of internal phase components is granulated with an aqueous solution of the binder obtained in Step 1 in a high-speed granulator.

### Step 3: Calibration of Granulate Before Drying

Before the drying process, the wet granulate is calibrated using a conical mill or oscillating granulator. The wet granulate can also be leveled manually using a steel sieve with an appropriate mesh size.

### Step 4: Drying

The granulate is dried using a fluid bed dryer or tray dryer at a targeted temperature of approximately 55° C. (40-65° C.) until the required weight loss after drying is achieved (0.2-3.0%).

Step 5: Calibration of Granulate after Drying

The dried granulate is leveled manually, on a steel sieve or using a conical mill or oscillating granulator.

Step 6: Mixing the Granulate with External Phase Components

All components of the external phase (except a lubricant) are mixed with the granulate in a low-speed mixer, with the speed adjusted to the size of the mixer tank and its filling. The lubricant is added at the end of the mixing stage.

### Step 7: Tableting

Tableting is performed separately for each dose of tablets comprising PKL-021 (doses are proportional, obtained by tableting to a specific targeted tablet weight).

Tableting is carried out on a rotary tablet press using appropriate equipment.

### Recipe for Tablets Comprising PKL-021

The concentration of PKL-021 in the product: from 10 to 40%.

Drug doses: proportional, obtained through targeted tablet weights.

TABLE 6
Recipe for tablets comprising PKL-021

| | Amount per |
|---|---|
| | 1 tablet |
| Component | [% w/w] |
| Internal phase (granulate) | |
| PKL-021 (active substance) | 10.0-40.0 |
| Binder (as a component of granulation liquid)* | 0.5-10.0 |
| Filling agent I | 10.0-50.0 |
| Filling agent II | 5.0-30.0 |
| Disintegrant | 0.0-10.0 |
| Purified water (as a component of granulation liquid)** | q.s. |
| External phase | |
| Filling agent III (type for direct tableting) | 0.0-30.0 |
| Disintegrant | 0.0-10.0 |
| Lubricant | 0.2-5.0 |
| Total | 100.0 |

*Added in the form of a 4-10% aqueous solution
**Removed during the granulate drying process

### REFERENCES

[1] Haq M., Shaeii A. E., Zervos E. E., Rosemurgy A. S.: In vitro and in vivo matrix metalloproteinase production by pancreatic cancer cells and by distant organs. Int. J. Surg. Invest. 2000, 1, 459-465.
[2] Collins H. M., Morris T. M., Watson S. A.: Spectrum of matrix metalloproteinase expression in primary and metastatic colon cancer: relationship to the tissue inhibitors of metalloproteinases and membrane type-1-matrix metalloproteinase. Br. J. Cancer 2001, 84, 1664-1670.
[3] Egeblad M. Werb Z.: New functions for the matrix metalloproteinases In cancer progression. Nat. Rev. Cancer. 2002, 2, 163-176.
[4] Rogowicz A., Zozulińska D., Wierusz- Wysocka B.: Znaczenie metaloproteinaz i ich inhibitorów w progresji naczyniowych powiktań cukrzycy- moźliwości terapeutyczne. Pol. Arch. Med. Wewn. 2007,117, 103-108.
[5] Barbara Pijet, Anna Konopka, Emilia Rejmak, Marzena Stefaniuk, Danylo Khomiak, Ewa Bulska, Stanislaw Pikul & Leszek Kaczmarek: The matrix metalloproteinase inhibitor marimastat inhibits seizures in a model of kainic acid-induced status epilepticus. Scientific Reports. 2020, 10:21314.
[6] Evans J. D., Stark A., Johnson C. D., Daniel F., Carmichael J., Buckels J., Imrie C. W., Brown P., Neoptolemos J. P.: A phase II trial of marimastat in advanced pancreatic cancer. Br. J. Cancer, 2001; 85: 1865-1870.
[7] Jones L. E., Humphreys M. J., Campbell F., Neoptolemos J. P., Boyd M. T.: Comprehensive analysis of matrix metalloproteinase and tissue inhibitor expression in pancreatic cancer: increased expression of matrix metalloproteinase-7 predicts poor survival. Clin. Cancer Res., 2004; 10: 2832-2845.
[8] Patten L. C., Berger D. H.: Role of proteases in pancreatic carcinoma. World J. Surg., 2005; 29: 258-263.
[9] Henrik S. Rasmussen i Peter P. McCann: Matrix Metalloproteinase Inhibition as a Novel Anticancer Strategy: A Review with Special Focus on Batimastat and Marimastat. Pharmacol. Ther. Vol. 75, No. 1, pp. 69-75, 1997.
[10] Leppert D., Lindberg R. L., Kappos L., Leib S. L.: Matrix metalloproteinases: multifunctional effectors of infl ammation in multiple sclerosis and bacterial meningitis. Brain Res. Brain Res. Rev., 2001; 36: 249-257.
[11] Yong V. W., Power C., Forsyth P., Edwards D. R.: Metalloproteinases in biology and pathology of the nervous system. Nat. Rev. Neurosci., 2001; 2: 502-511.

1. A process for the preparation of marimastat, characterized in that the process includes the following steps:
a) (2S,3R)-2-hydroxy-3-isobutylsuccinic acid is reacted in the environment of 2,2-dimethoxypropane with the addition of p-toluenesulfonic acid, pyridinium p-toluenesulfonate or 10-camphorsulfonic acid at a temperature of 35-55° C. for 15-30 h, then after the reaction is completed, a tertiary amine selected from the group comprising diisopropylethylamine, triethylamine and N-methylmorpholine is added to obtain (R)-2-((S)-2,2-dimethyl-5-oxo-1,3-dioxolan-4-yl)-4-methylpentanoic acid;
b) (R)-2-((S)-2,2-dimethyl-5-oxo-1,3-dioxolan-4-yl)-4-methylpentanoic acid is reacted with (S)-2-amino-N,3,3-trimethylbutanamide or its hydrochloride in the environment of 2-(1H-benzotriazole-1-yl)-1,1,3,3-tetramethylaminium tetrafluoroborate and a tertiary amine selected from the group comprising diisopropylethylamine, triethylamine and N-methylmorpholine in acetonitrile or methylene chloride, then the reaction mixture is subjected to an extraction process to obtain crude (R)-N-((S)-3,3-dimethyl-1-methylamino-1-oxobutan-2-yl)-2-((S)-2,2-dimethyl-5-oxo-1,3-dioxolan-4-yl)-4-methylpentanamide, which is then used in step c) or macerated at boiling in methyl tert-butyl ether or isopropyl ether for 1-3 h, and then at room temperature for 15-30 h to obtain (R)-N-((S)-3,3-dimethyl-1-methylamino-1-oxobutan-2-yl)-2-((S)-2,2-dimethyl-5-oxo-1,3-dioxolan-4-yl)-4-methylpentanamide; and
c) (R)-N-((S)-3,3-dimethyl-1-methylamino-1-oxobutan-2-yl)-2-((S)-2,2-dimethyl-5-oxo-1,3-dioxolan-4-yl)-4-methylpentanamide or crude (R)-N-((S)-3,3-dimethyl-1-methylamino-1-oxobutan-2-yl)-2-((S)-2,2-dimethyl-5-oxo-1,3-dioxolan-4-yl)-4-methylpentanamide is reacted with a 50% aqueous solution of hydroxylamine in a solvent selected from a lower carboxylic acid ester, a non-polar solvent or a polar solvent, then acetone is added and stirred at boiling for 0.5-1 h, then the reaction mixture is subjected to azeotropic distillation process in order to remove water, and then stirred at room temperature for 15-30 h to obtain marimastat.

2. The process according to claim 1, characterized in that the reaction in step b) is carried out at a temperature from −10° C. to room temperature.

3. The process according to claim 2, characterized in that the reaction in step b) is carried out for 1-24 h.

4. The process according to claim 1, characterized in that the reaction in step c) with the 50% aqueous solution of hydroxylamine is carried out at a temperature from room temperature to boiling point.

5. The process according to claim 4, characterized in that the reaction in step c) with the 50% aqueous solution of hydroxylamine is carried out for 1-30 h.

6. The process according to claim 1, characterized in that the lower carboxylic acid ester is selected from the group comprising ethyl acetate and isopropyl acetate.

7. The process according to claim 1, characterized in that the non-polar solvent is selected from the group comprising 2-methyltetrahydrofuran, 1,2-dimethoxyethane, methylene chloride and chloroform.

8. The process according to claim 1, characterized in that the polar solvent is selected from the group comprising acetonitrile and isopropyl alcohol.

9. The process according to claim 1, characterized in that the tertiary amine in step b) is added in two portions, the first portion before adding (S)-2-amino-N,3,3-trimethylbutanamide or its hydrochloride, the second portion after adding (S)-2-amino-N,3,3-trimethylbutanamide or its hydrochloride.

10. The process according to claim 1, characterized in that the tertiary amine is diisopropylethylamine.

11. The process according to claim 1, characterized in that during the azeotropic distillation process in step c), the volume of distilled solvent is replenished by successive addition of further portions of solvent.

12. Marimastat prepared by the process defined in claim 1.

13. A pharmaceutical composition characterized in that it comprises marimastat defined in claim 12 and a pharmaceutically acceptable carrier.

14. The composition according to claim 13, characterized in that it is in the form of a tablet, modified-release tablet, pill, capsule, powder, granules, pellets, suspension, emulsion, solution, oral liquid forms, e.g. syrup, solution or suspension for injection, solution for infusion, eye drops, ointment, gel, suppository, globule or therapeutic system, e.g. implant, vaginal ring, nanofiber.

15. The pharmaceutical composition defined in claim 13 for use as a medicament.

16. The pharmaceutical composition defined in claim 13 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

17. Marimastat defined in claim 12 for use as a medicament.

18. Marimastat defined in claim 12 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.
